(19) United States
(12) Patent Application Publication
(10) Pub. No.: US 2014/0355001 A1
(43) Pub. Date: Dec. 4, 2014

(54) Measuring Deflection in an Optical Fiber Sensor by Comparing Current and Baseline Frames of Speckle Interference Patterns

(71) Applicant: Stratus Devices, Inc., Sunnyvale, CA (US)

(72) Inventor: Charles J. Kring, Sunnyvale, CA (US)

(73) Assignee: Stratus Devices, Inc., Sunnyvale, CA (US)

(21) Appl. No.: 13/903,854

(22) Filed: May 28, 2013

(51) Int. Cl. G01D 5/353; A61B 5/024; A61B 5/08

(52) U.S. Cl. CPC G01D 5/35329; A61B 5/0816; A61B 5/02444
USPC 356/482; 600/476; 600/479

(57) ABSTRACT

A sensor compares frames of pixels representing a speckle pattern caused by interference of light through an optical fiber to detect magnitudes of deflection of the fiber. A coherent light source illuminates the optical fiber. An image sensor captures the speckle pattern and frames of pixels produced by the image sensor are processed to determine deflection. A baseline frame is generated from frames previously received. Each frame is compared to the baseline frame to determine the cumulative amount of deflection on the fiber. To compensate for drift and large-scale movements of the optical fiber, the baseline frame is updated as frames are received. The processed output from comparing to the baseline frame has larger amplitude signals than from comparing to adjacent frames due to larger deflections over time since the baseline frame. Signal-to-Noise ratio is improved, and the processed output better matches a plot of the actual total deflection.

### FIELD OF THE INVENTION

This invention relates to sensing devices, and more particularly to detecting changes in interference patterns from fiber optics.

### BACKGROUND OF THE INVENTION

Optical fibers are sometimes used to detect physical movement, deflection, or perturbation, such as bending of a security fence or of a mat with a fiber optics strand placed underneath a patient in a bed. Coherent light is used to illuminate the fiber at one end, while a photo detector at the other end reads an interference, stipple, or speckle pattern. The speckle pattern is created by multi-mode interference in the long optical fiber. This speckle pattern changes as the fiber is deflected.

FIG. 1 shows a prior-art multi-mode optical-fiber sensor. Coherent light source 101 illuminates one end of optical fiber 102. The light exiting the other end of optical fiber 102 produces modal interference pattern 103, which is commonly called a speckle pattern. Interference pattern 103 is detected by image sensor or photo-detector 104 to produce a series of readings 105 that are processed by image processor 106 to produce processed output 107.

A multi-mode optical-fiber sensor may be used as an intrusion alarm system. A subset of the speckle pattern may be detected. The overall amount of light detected changes as the speckle pattern changes. The light detected may be compared against the previous reading to generate the sensor output.

When an image sensor is used rather than a photo detector, a frame of pixels may be detected and generated from the speckle pattern. Subsequent video frames from the image sensor may be compared to detect changes in the speckle pattern. Sometimes only a portion of the speckle pattern or frame is detected or processed.

The speckle pattern is not always used in detection. Interferometry between two optical fibers may be used to detect patient vital signs or as a security device for perimeter protection. A much simpler photo-detector may be used when the speckle pattern is not detected.

Multi-mode fiber-optic sensors are distinguished by whether the speckle pattern is detected with a photo-detector, that only produces a single overall output, or with a Charge-Coupled Device (CCD), complementary metal-oxide-semiconductor (CMOS) sensor, or some other 2-D image sensor that produces a frame of pixel values. Fiber-optic sensors based upon image sensors tend to be significantly more sensitive than sensors based upon photo detectors because small displacements within the field of view of the photo detector do not register as a change in the overall amount of light received by the photo detector, whereas such small displacements in the speckle pattern can be detected on a 2-D image sensor that produces an array of pixel readings.

Existing multi-mode fiber-optic sensors based upon image sensors typically compare subsequent speckle images to detect changes to the speckle pattern. In FIG. 1, changes over time to speckle pattern 103 are detected by image sensor 104 that produces a 2-D frame of pixels for each snapshot in time. Over a longer period of time, a stream of frames of pixels, readings 105, may be processed by image processor 106 to produce processed output 107.

FIG. 2 shows image processing on a frame-by-frame basis. Incoming frames 200 from the image sensor are processed on a frame-by-frame basis and delayed by frame delay 206. Current frame 201 is compared with previous frame 202 on a pixel-by-pixel basis, such as by subtraction of pixel values by pixel comparator 203. Processed output 107 is the sum of the absolute value of the difference between each pixel and the corresponding pixel in the previous frame. Summer 205, absolute value generator 204, and pixel comparator 203 produce a sum-of-the-absolute difference (SAD) for all pixels between current frame 201 and previous frame 202.

FIG. 3 shows plots of raw and processed changes to a speckle pattern. Dotted curve 301 is a representation of the cumulative change to the speckle pattern over time, such as for changes in readings 105 from the image sensor. Frames are sampled periodically, so for every sample there is delay 302, represented by a horizontal line, and sensor reading 303, represented by a vertical step. In this figure, sensor reading 303 indicates the absolute value of difference between the corresponding pixel in a frame and in the previous frame summed across all pixels. It is the measured deflection of the optical fiber during the time period of delay 302.

Processed output 107, shown as bar readings 304 in the lower graph, consists individual sensor readings 303 over time. Note that the shape of the processed output's bar readings 304 is not consistent with the change to the speckle pattern of dotted curve 301 because individual readings 303 measure the amount of change between frames. In fact, when cumulative change to the speckle pattern reaches a peak, such as at the middle peak of dotted curve 301 (top graph), processed output 107 has bar reading 304 that are close to zero (middle of bottom graph).

Since the individual readings can drop to almost zero at the peak of the cumulative change to the speckle pattern, periodic signals such as breathing or vibration may be destroyed because every peak on the signal is transformed into multiple peaks in processed output 107. This complicates using a FFT or a DFT to take a frequency response for determining breath rate, heart rate, or frequency of vibration.

Furthermore, since individual reading 303 measures the incremental change between subsequent frames, every reading 303 is less than the cumulative change to the speckle pattern, shown by dotted curve 301. Hence the peak output of processed output 107 is less than the peak change to the speckle pattern, the peak of dotted curve 301, reducing the signal to noise ratio of the sensor.

If the sampling frequency is increased, the sample period decreases and the difference between subsequent frames is reduced. FIG. 4 shows plots of raw and processed changes to a speckle pattern for a higher sampling frequency.

At a higher sampling frequency, delay 302 (FIG. 3) is reduced to delay 402. Note that sensor reading 403 is smaller than corresponding sensor reading 303 because the change to the speckle pattern, shown by dotted curve 301, is smaller because of the smaller sample period. Note also that processed output 107 has bar readings 404 that are much smaller than bar readings 304 of FIG. 3. Thus increasing the sampling frequency decreases amplitude and the signal-to-noise ratio of the sensor.

What is desired is better processing of frames of pixels from image sensors for optical fiber deflection detectors. A better signal-to-noise ratio is desired for the processed output of such detectors. A more direct and accurate measure of fiber deflection is desirable.

### BRIEF DESCRIPTION OF THE DRAWINGS

FIG. 1 shows a prior-art multi-mode optical-fiber sensor.

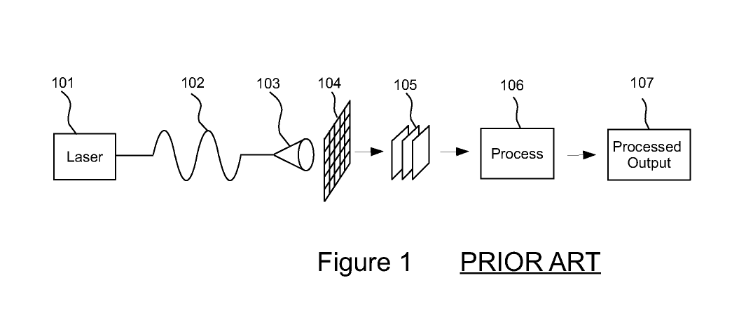

FIG. 2 shows image processing on a frame-by-frame basis.

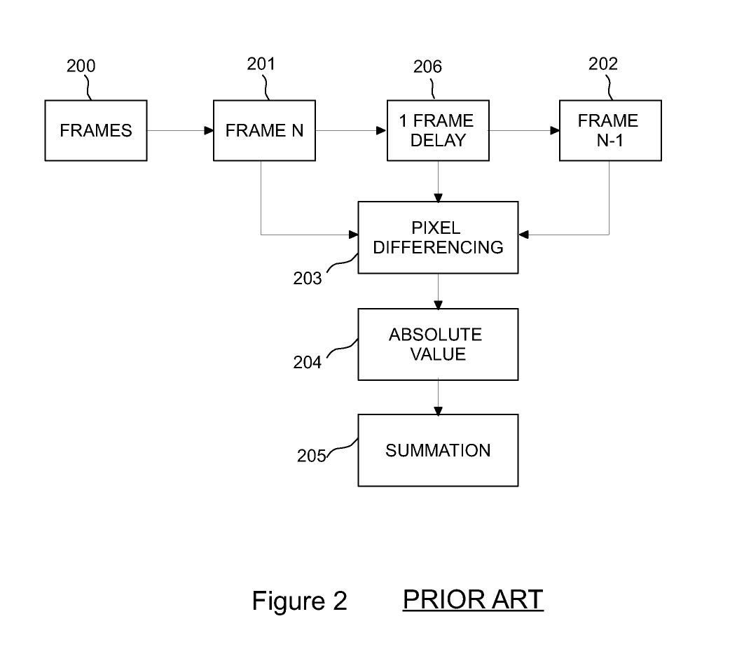

FIG. 3 shows plots of raw and processed changes to a speckle pattern.

FIG. 4 shows plots of raw and processed changes to a speckle pattern for a higher sampling frequency.

FIG. 5 shows a baseline-comparing multi-mode optical-fiber sensor.

FIG. 6 shows processing frames against a baseline image frame.

FIG. 7 shows plots of raw and processed changes to a speckle pattern using baseline frame comparison.

FIG. 8 shows plots of raw and processed changes to a speckle pattern for a higher sampling frequency using baseline comparison.

FIG. 9 shows an updating-baseline multi-mode optical-fiber sensor.

FIG. 10 shows poor baseline management.

FIG. 11 shows processing frames against a baseline image frame with baseline updating.

FIG. 12 shows a baseline update process.

FIG. 13 is a graph of a processed output and a raw adjacent frame difference over time.

### DETAILED DESCRIPTION

The present invention relates to an improvement in optical-fiber deflection detectors. The following description is presented to enable one of ordinary skill in the art to make and use the invention as provided in the context of a particular application and its requirements. Various modifications to the preferred embodiment will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed.

The inventor has developed a method for processing frames received from the image sensor in a multi-mode optical-fiber sensor to more directly measure deflection. Rather than compare adjacent frames, frames of pixels are compared against a baseline frame to produce a processed output that is consistent with cumulative changes to the speckle pattern, has an optimal signal to noise ratio, and that does not depend upon the sampling rate.

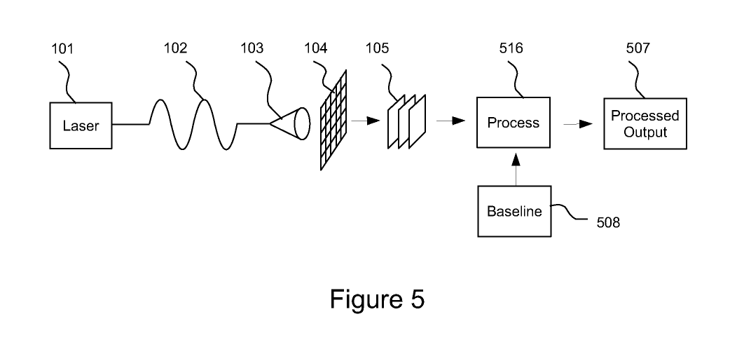

FIG. 5 shows a baseline-comparing multi-mode optical-fiber sensor. Coherent light source 101 illuminates one end of optical fiber 102. The light exiting the other end of optical fiber 102 produces modal interference pattern 103, which is commonly called a speckle pattern. Interference pattern 103 is detected by image sensor 104 to produce a series of readings 105 or frames that are each compared to baseline image 508 by baseline image processor 516 to produce processed output 507.

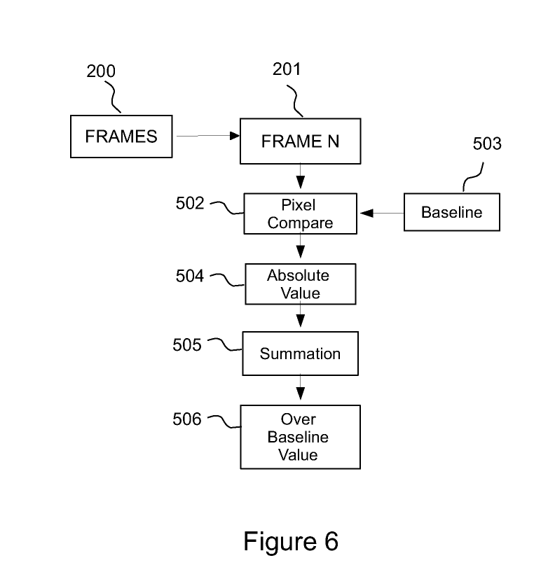

FIG. 6 shows processing frames against a baseline image frame. Incoming frames 200 from the image sensor are processed and compared to baseline frame 503 on a frame-by-frame basis. Current frame 201 is compared with baseline frame 503 on a pixel-by-pixel basis, such as by subtraction of pixel values by pixel comparator 502. Processed output 507 (FIG. 5) is over-baseline value 506, which is the sum of the absolute value of the difference between each pixel in the current frame and the corresponding pixel in the baseline frame. Summer 505, absolute value process 504, and pixel comparator 502 produce a sum-of-the-absolute difference (SAD) for all pixels between current frame 201 and baseline frame 503. This SAD is not the difference between adjacent or sequential frames, but instead is the difference of the current frame to the baseline frame, over-baseline value 506.

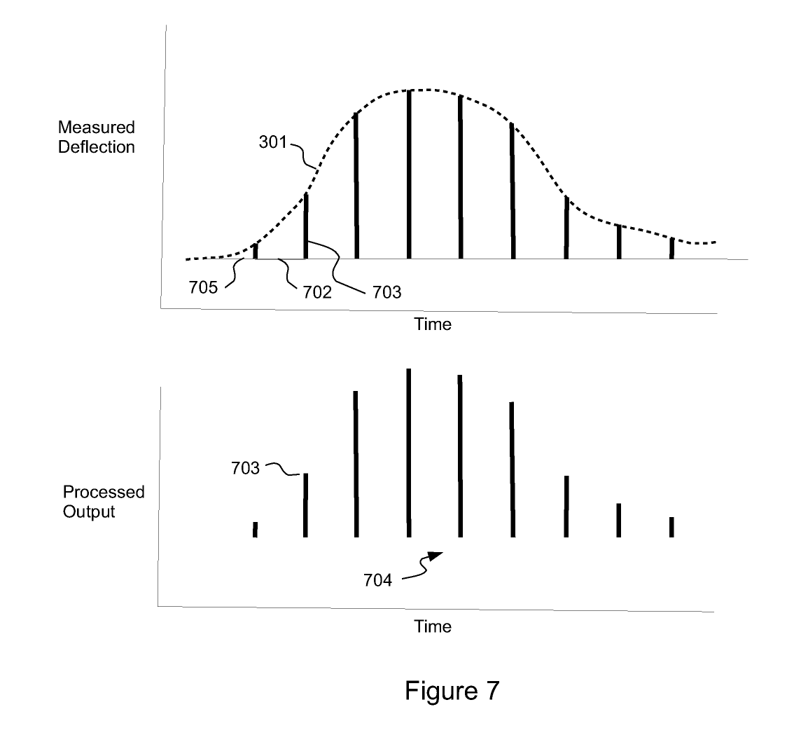

FIG. 7 shows plots of raw and processed changes to a speckle pattern using baseline frame comparison. In this Figure, baseline frame 705 is selected to be the first frame received from the image sensor and every subsequent frame is compared to baseline frame 705. Since baseline frame 705 is not compared to any prior frame, it has no difference or SAD with another frame and is represented by the horizontal base line.

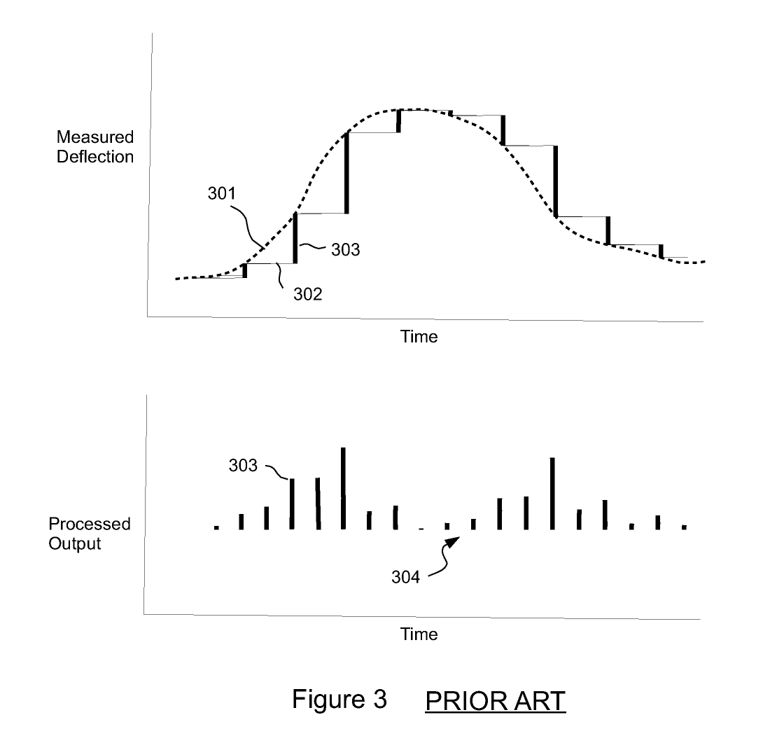

Dotted curve 301 is a representation of the cumulative change to the speckle pattern over time, the cumulative measured deflection of readings 105 from the image sensor. Frames are sampled periodically, so for every sample there is delay 702, represented by a horizontal line segment, and over-baseline reading 703, represented by a vertical step. In this figure, over-baseline reading 703 indicates the absolute value of difference between the corresponding pixel in a frame and in the baseline frame summed across all pixels. It is the measured deflection of the optical fiber during all time periods since the baseline frame. Thus the height of over-baseline readings 703 tend to be much larger than sensor readings 303 (FIG. 3), since the over-baseline readings represent the total deflection of the fiber rather than the incremental deflection that occurred between frames.

Processed output 507 (FIG. 5), which is over-baseline value 506 (FIG. 6), is shown as bar readings 704 in the lower graph, and consists of over-baseline readings 703 generated over time. Note that the shape of the processed output's processed readings 704 is consistent with the cumulative change to the speckle pattern of dotted curve 301 because individual over-baseline readings 703 measure the amount of change between a frame and the baseline frame, not adjacent frame. When cumulative change to the speckle pattern reaches a peak, such as at the middle peak of dotted curve 301 (top graph), processed output 507 has processed readings 704 that are also at a peak. The shape of the envelope of processed readings 704 matches the shape of dotted curve 301.

FIG. 7 shows that processed readings 704 are consistent with changes to the speckle pattern (dotted curve 301). Also, the amplitude of processed readings 704 is about the same as the amplitude of cumulative changes to the speckle pattern shown by dotted curve 301. Processed readings 704 track changes to the speckle pattern. The signal-to-noise ratio of the sensor is optimal since over-baseline readings 703 have a large amplitude, as shown by the large heights. Periodic signals such as breathing or heartbeat may be accurately captured and easily processed using standard discreet signal post-processing algorithms.

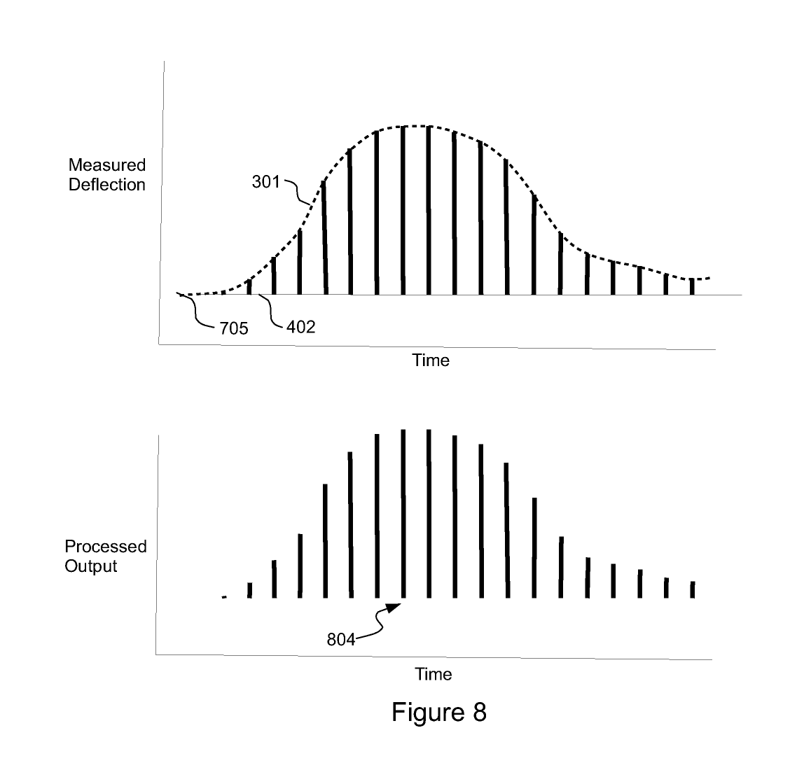

FIG. 8 shows plots of raw and processed changes to a speckle pattern for a higher sampling frequency using baseline comparison. If the sampling frequency is increased, the sample period decreases and the difference between subsequent frames is reduced. However, since each frame is compared to a baseline frame, not to adjacent frame, the time delay between frames is not critical.

At a higher sampling frequency, delay 702 (FIG. 7) is reduced to delay 402. Although frame-to-frame differences are smaller due to the smaller time delay between frames, the difference to the baseline frame is not affected by the delay between frames and the insertion of additional frames for the higher sampling frequency.

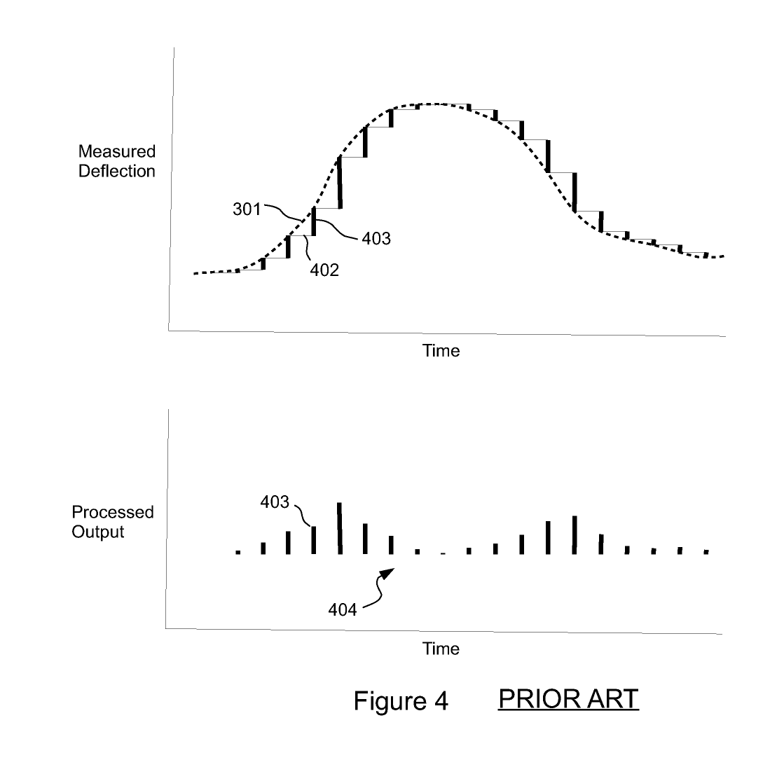

Processed readings 804 are shown assuming the same delay 402 as in FIG. 4 and cumulative changes in the speckle pattern as shown by dotted curve 301. Baseline frame 705 is selected to be the first frame received from the image sensor. Note that the amplitude of the processed readings 804 is the same as the amplitude of changes to the speckle pattern, dotted curve 301. The signal-to-noise ratio of the sensor is optimal.

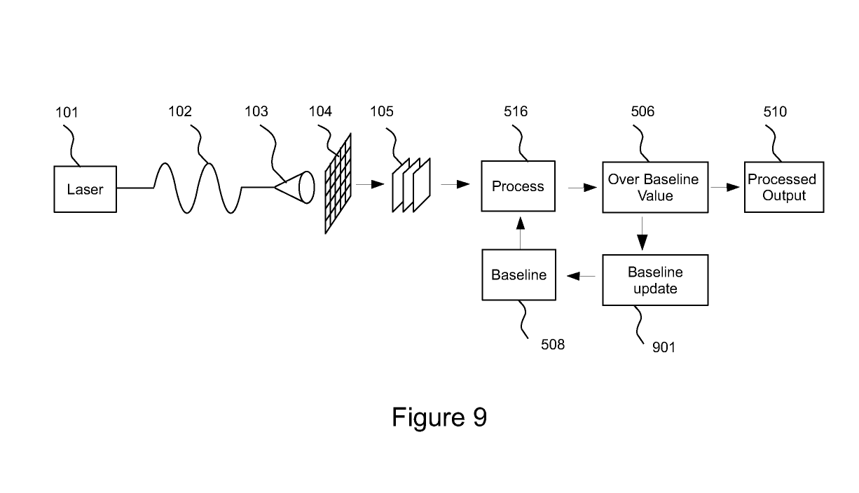

FIG. 9 shows an updating-baseline multi-mode optical-fiber sensor. Coherent light source 101 illuminates one end of optical fiber 102. Constructive and destructive interference of the light as it propagates through the fiber creates modal interference pattern 103, a speckle pattern, that changes as the fiber is deflected. Changes to the speckle pattern are detected by image sensor 104 to produce a series of frames or readings 105 that are each compared to baseline image 508 by baseline image processor 516 to generate over-baseline value 506. Over-baseline value 506 is output as processed output 510.

Baseline image 508 is periodically updated based upon the sensor output to compensate for dark-current noise in the image sensor, changes in geometry of the optical fiber, and other sources of sensor drift. Baseline updater 901 reads over-baseline value 506 and adds a weighted amount of over-baseline value 506 and the old value of baseline image 508 to generate a new updated value for baseline image 508.

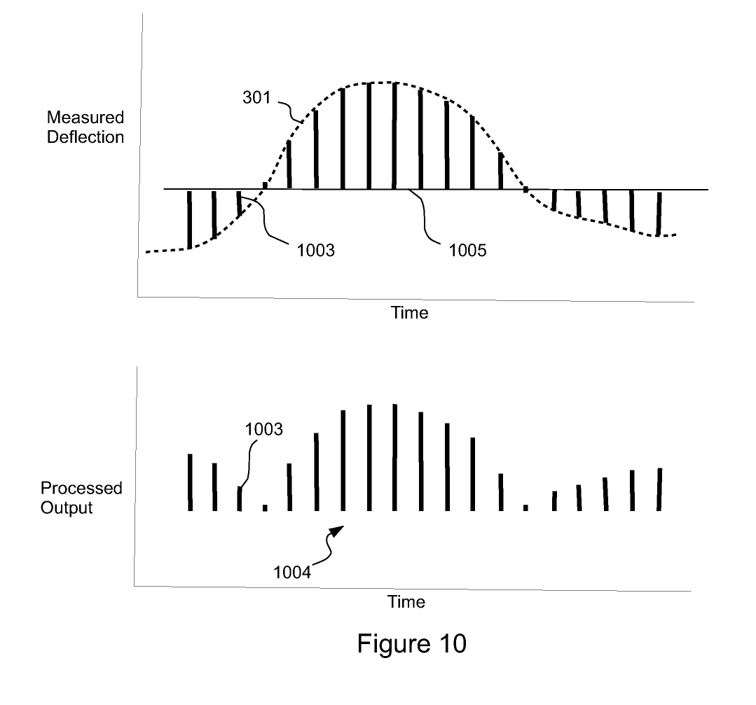

FIG. 10 shows poor baseline management. In this example, baseline image 1005 (the horizontal line) is selected to be a frame intermediate to the minimum and maximum of changes to the speckle pattern. This can occur with a periodic signal if the baseline frame is incorrectly selected. Since the absolute difference is obtained by image processor 516 , below-baseline values of over-baseline readings 1003 are reflected along the axis of baseline image 1005 so that the processed output has large positive bars rather than large negative bars for these below-baseline readings.

Because of this reflection of below-baseline readings, over-baseline readings 1003 of the processed output do not match changes to the speckle pattern, dotted curve 301, and the signal-to-noise ratio of the sensor is reduced. In fact, processed output 1004 is not improved over sensor output 304 from the prior art.

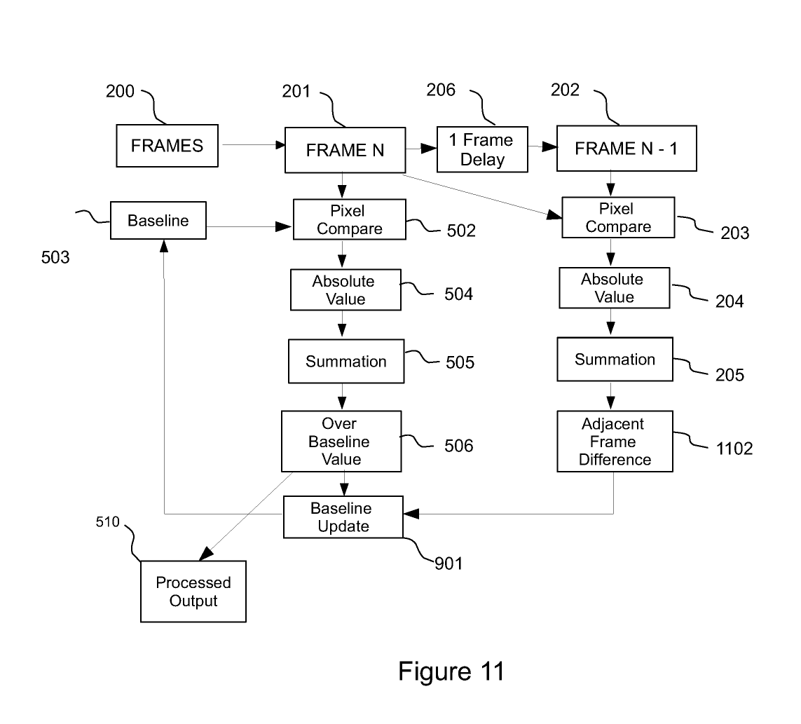

FIG. 11 shows processing frames against a baseline image frame with baseline updating. Incoming frames 200 from the image sensor are processed and compared to baseline frame 503 on a frame-by-frame basis. Current frame 201 is compared with baseline frame 503 on a pixel-by-pixel basis, such as by subtraction of pixel values by pixel comparator 502. Processed output 510 (FIG. 9) is over-baseline value 506, which is the sum of the absolute value of the difference between each pixel in the current frame and the corresponding pixel in the baseline frame. Summer 505, absolute value process 504, and pixel comparator 502 produce a sum-of-the-absolute difference (SAD) for all pixels between current frame 201 and baseline frame 503. This SAD is not the difference between adjacent or sequential frames, but instead is the difference of the current frame to the baseline frame, over-baseline value 506.

Incoming frames 200 from the image sensor are processed on a frame-by-frame basis and delayed by frame delay 206. Current frame 201 is compared with previous frame 202 on a pixel-by-pixel basis, such as by subtraction of pixel values by pixel comparator 203. Adjacent frame difference 1102 is the sum of the absolute value of the difference between each pixel and the corresponding pixel in the previous frame. Summer 205, absolute value generator 204, and pixel comparator 203 produce a sum-of-the-absolute difference (SAD) for all pixels between current frame 201 and previous frame 202.

Baseline updater 901 reads adjacent frame difference 1102 and determines when to update baseline frame 503, and by how much.

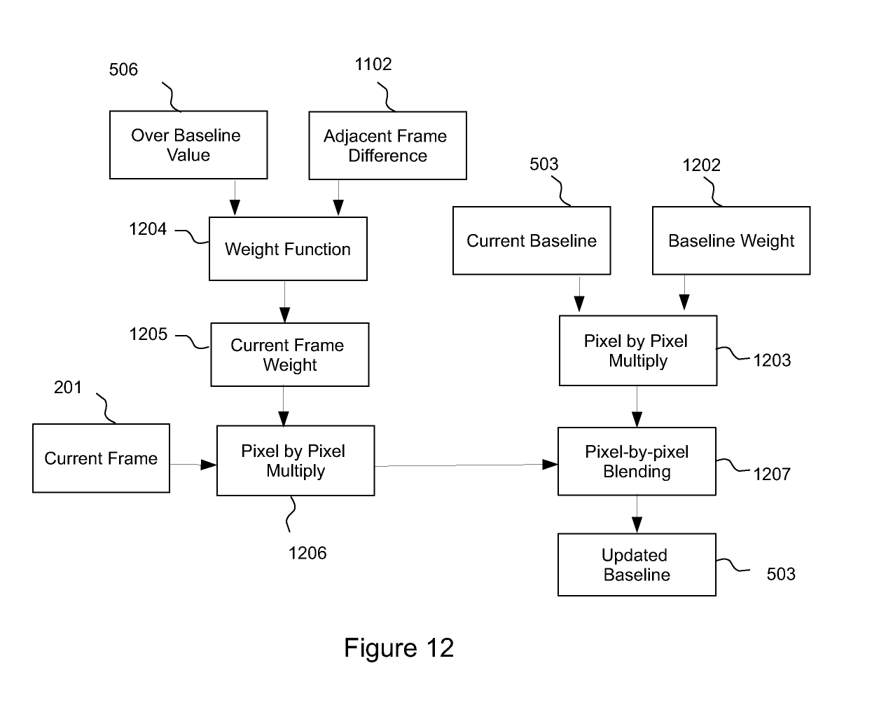

FIG. 12 shows a baseline update process. The current or a recent over-baseline value 506 and adjacent frame difference 1102 are evaluated by current frame weight function 1204 to produce current frame weight 1205. If the current frame weight is greater than 0, pixels in baseline frame 503 are multiplied in multiplier 1203 by baseline weight 1202 and pixels in the current frame 201 are multiplied in multiplier 1206 by current frame weight 1205. Corresponding weighted pixels in the baseline frame and in the current frame are added by pixel blender 1207 to produce updated baseline frame 503. In practice, weights are typically generated from a user-specified parameter. Current frame weights may range from 0 to 100%, while the baseline frame weight is typically 80-95%. This weighting results in a blending of current frame 201 with previous baseline frame 503.

Weight function 1204 is dependent upon the particular application that the sensor is used for. For example, in security monitoring applications, the sensor is normally quiet with occasional perturbations that need to be reported. In patient monitoring applications, the sensor is normally not quiet but rather has to monitor a repeating signal, such as from a person's respiration.

A variety of baseline weighting functions are possible and different functions may be preferred in different circumstances. Multiple baseline weighting functions 1204 can be compiled and used. Each baseline weight function may be tested in a priority order and the result of the first baseline weight function that returns a non-zero value is selected for use.

Frame weighting function 1204 may monitor adjacent frame difference 1102 over a sliding window of time to determine a representative adjacent frame difference that indicates that the sensor is not currently excited by external stimuli. This identifies periods of time when the sensor is quiet, such as when an intrusion alarm is active but there is no activity. During quiet periods, adjacent frame difference 1102 is lower than the representative adjacent frame difference and the current frame is heavily weighted in the baseline image. When the sensor is not quiet, adjacent frame difference 1102 is greater than the representative internal reading and the current frame is given no weight in the baseline image.

Another possible baseline weighting function 1204 monitors over baseline value 506 and keeps track of large minima in the reading over time. Frames corresponding to these minima are heavily weighted in the baseline image and other frames are not. This tends to add frames at the bottom of a repeating signal to the baseline image and produces an optimal baseline when the sensor is actively monitoring a signal such as respiration.

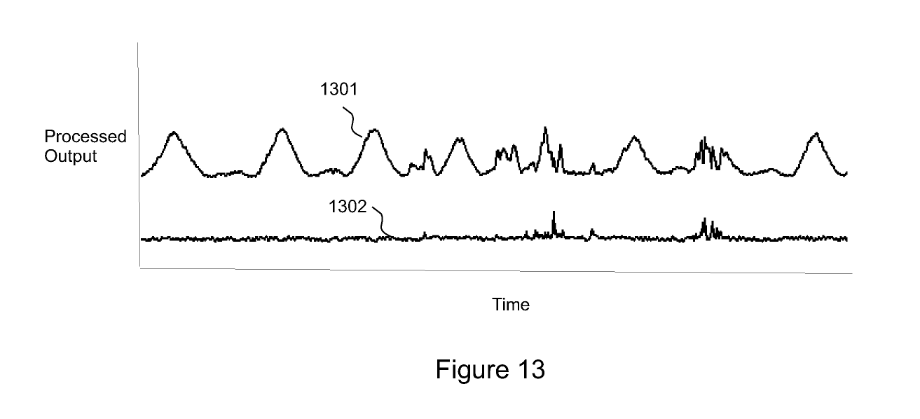

FIG. 13 is a graph of a processed output and a raw adjacent frame difference over time. Curve 1302 shows the adjacent frame difference signal, which has small variations since frame-to-frame changes tend to be relatively small, especially for higher sampling frequencies. The small amplitude of signals in curve 1302 also produces a small signal-to-noise ratio.

Curve 1301 shows a processed output signal that is generated by comparing each frame to a baseline frame. The baseline frame may be updated as needed. Since the differences in the speckle pattern are large for a current frame that is a relatively long distance in time from the baseline frame, a large amplitude signal is generated. This large signal has a better signal-to-noise ratio. Periodic variations due to real monitored behavior, such as breathing or vibrations of a security fence due to wind are visible in curve 1301. Further post-processing, such as by a digital-signal processor (DSP) may be performed. For example, a Fast Fourier Transform (FFT) may be used to extract the breathing rate from the periodic peaks in curve 1301.

### Alternate Embodiments

Several other embodiments are contemplated by the inventor. For example the output from the image sensor could be an array of pixel values of various pixel formats such as intensity or color. While each frame has been described as being compared to the baseline frame, only a subset of frames could be compared, such as every other frame, or every third frame, etc.

Over baseline processing and baseline processing can occur on a subset of the pixels produced by the image sensor. Multiple fibers can point at different zones in the image sensor and each zone could be processed separately.

The adjacent frame difference could run a slower frame rate than the over-baseline processing. At high frame rates the adjacent frame difference gets very small. Baseline update might per performed at a lower frame rate than the over-baseline processing.

The processed output could be further post-processed, such as by a FFT, a Discrete Fourier Transform (DFT), or a wavelet transform to determine the rate of periodic signals. This can be used to determine heartbeat or breath rate in patient monitoring, or to determine frequency of vibration.

The image sensor can be a CMOS image sensor, a CCD sensor or any other pixel-based image sensor. The light source may support multiple frequencies. For example, a combination of red, green and blue and/or Infrared lasers can be used as the light source.

The particular image processed might not be the current image. For example, a baseline update might be against the current frame and the processed output against a previous frame. Registers may be added for pipelining or delaying operations.

There are a large number of possible variations of weight function 1204. Functions and processes may be performed by programming a general-purpose computer, or by dedicated hardware functions, firmware, or various combinations. The weight function can depend upon other factors such as a FFT or DFT transform of the processed output.

While a sum-of-the-absolute difference (SAD) function has been described for comparing pixels, other compare functions could be used. Frames could be histograms of the number of pixels with a particular value. Pixels could be grouped (e.g. a ‘pixel’ could be a 4×4 patch of pixels from the image sensor.

The background of the invention section may contain background information about the problem or environment of the invention rather than describe prior art by others. Thus inclusion of material in the background section is not an admission of prior art by the Applicant.

Any methods or processes described herein are machine-implemented or computer-implemented and are intended to be performed by machine, computer, or other device and are not intended to be performed solely by humans without such machine assistance. Tangible results generated may include reports or other machine-generated displays on display devices such as computer monitors, projection devices, audio-generating devices, and related media devices, and may include hardcopy printouts that are also machine-generated. Computer control of other machines is another tangible result. Patient monitors, automatic generation of patient records, automatic alarms that are triggered when breathing or heart beat stops or is irregular (e.g. baby monitor) are other examples of tangible results.

Any advantages and benefits described may not apply to all embodiments of the invention. When the word “means” is recited in a claim element, Applicant intends for the claim element to fall under 35 USC Sect. 112, paragraph 6. Often a label of one or more words precedes the word “means”. The word or words preceding the word “means” is a label intended to ease referencing of claim elements and is not intended to convey a structural limitation. Such means-plus-function claims are intended to cover not only the structures described herein for performing the function and their structural equivalents, but also equivalent structures. For example, although a nail and a screw have different structures, they are equivalent structures since they both perform the function of fastening. Claims that do not use the word “means” are not intended to fall under 35 USC Sect. 112, paragraph 6. Signals are typically electronic signals, but may be optical signals such as can be carried over a fiber optic line.

The foregoing description of the embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

I claim:

1. A multi-mode fiber-optic sensor comprising:
an image processor that receives a speckle pattern from an image sensor, the speckle pattern created by interference of light passing through an optical fiber, wherein deflection of the optical fiber changes the speckle pattern, the image processor outputting a current frame of pixels for each sample period, wherein the current frame is in a sequence of frames;
a baseline frame representing an array of pixel values;
a frame comparator that produces an over-baseline value that represents a difference between the current frame and the baseline frame; and
a processed output that outputs the over-baseline value for current frames in the sequence of frames.

2. The multi-mode fiber-optic sensor of claim 1 wherein the baseline frame is representative of when the optical fiber is not deflected.

3. The multi-mode fiber-optic sensor of claim 1 wherein the baseline frame is a minimum frame in the sequence of frames, the minimum frame representative of a bottom of a breathing cycle or of a heart beat.

4. The multi-mode fiber-optic sensor of claim 1 further comprising:
a baseline updater that replaces the baseline frame with an updated baseline frame while the sequence of frames is being processed.

5. The multi-mode fiber-optic sensor of claim 1 wherein the baseline frame is a composite frame generated from frames in the sequence of frames.

6. The multi-mode fiber-optic sensor of claim 5 wherein a contribution of individual frames to the baseline frame depends upon a magnitude of difference between the speckle pattern of a frame and the speckle pattern of the baseline frame.

7. The multi-mode fiber-optic sensor of claim 4 further comprising:
a prior frame representing an array of pixel values for a frame before the current frame in the sequence of frames; and
an adjacent frame comparator that produces an adjacent frame difference value that represents a difference between the prior frame and the current frame.

8. The multi-mode fiber-optic sensor of claim 7 further comprising:
a current weight generator, receiving the adjacent frame difference value and the over-baseline value, for generating a current weight for the current frame;
a pixel blender that blends pixels in the current frame into the baseline frame depending upon the current weight.

9. The multi-mode fiber-optic sensor of claim 8 wherein the current weight generator;
further comprises:
a baseline pixel multiplier for multiplying pixels in the baseline frame with a baseline weight to generate weighted baseline pixels for the baseline frame; and
a pixel combiner for adding the weighted current pixels to the weighted baseline pixels to generate blended pixels;
wherein the blended pixels are stored as updated pixels for the baseline frame,
wherein the baseline frame is updated by the updated pixels generated by weighted multiplication and blending.

10. The multi-mode fiber-optic sensor of claim 4 further comprising:
a coherent light source for generating a coherent light;
a fiber optic strand having a first opening receiving the coherent light, and a second opening, the fiber optic strand forming the optical fiber;
an image sensor that receives light exiting the second opening of the fiber optic strand, the light forming a speckle pattern created by interference in the fiber optic strand;
wherein the fiber optic strand is deformed by a monitored movement;
wherein the processed output is a measure of a cumulative magnitude of the monitored movement since a baseline frame.

11. The multi-mode fiber-optic sensor of claim 1 further comprising:
a pixel comparator for comparing pixels from the current frame to corresponding pixels in the baseline frame to generate pixel differences;
an absolute generator for generating absolute pixel differences which are absolute values of the pixel differences from the pixel comparator;
a summer for summing the absolute pixel differences from the absolute generator to generate a sum-of-the-absolute differences (SAD), the SAD being an over-baseline value that indicates a magnitude of differences between the speckle pattern of the current frame and a speckle pattern of the baseline frame.

12. The multi-mode fiber-optic sensor of claim 11 further comprising:
a physical memory for storing a plurality of frames of pixels generated by the image sensor.

13. The multi-mode fiber-optic sensor of claim 12 further comprising:
a processor for executing instructions, the processor executing routines for generating the sum-of-the-absolute difference (SAD) from pixel values read from the physical memory.

14. A deflection sensor comprising:
an image sensor that generates a frame of pixels for each sampling period, the frame of pixels representing an interference pattern created by deflection of light in an optical fiber;
a first frame memory for storing a current frame of pixels from the image sensor;
a baseline frame memory for storing a baseline frame of pixels, the baseline frame not being an adjacent frame that is immediately adjacent to the current frame in a sequence of frames;
an image processor, coupled to the first frame memory and to the baseline frame memory, for comparing each pixel in the first frame memory with a corresponding pixel having a same x,y location in the baseline frame of pixels as in the current frame of pixels, and generating an overall frame difference value that is output as an over-baseline value;
a processed output that outputs the over-baseline value from the image processor for each sampling period;
whereby the current frame is compared to the baseline frame rather than to an adjacent frame.

15. The deflection sensor of claim 14 further comprising:
a baseline updater that updates pixels in the baseline frame memory;
wherein the baseline updater updates the pixels in the baseline frame memory with pixels from the current frame

16. The deflection sensor of claim 14 wherein the overall frame difference value is a sum-of-the-absolute difference (SAD);
wherein the image processor comprises:
a pixel summer for generating a pixel difference for each pixel x,y location in the current frame of pixels;
an absolute generator for generating an absolute value of the pixel difference; and
a final summer for adding together the absolute values for all pixel x,y locations in the current frame.

17. The deflection sensor of claim 14 wherein an envelope bounding outputted readings of the processed output has a same shape as a waveform representing total cumulative deflection of the optical fiber, wherein minima of the envelope occur coincident in time with minima of the waveform representing total cumulative deflection of the optical fiber, and maxima of the envelope occur coincident in time with maxima of the waveform representing total cumulative deflection of the optical fiber.

18. The deflection sensor of claim 17 further comprising:
a post-processor, receiving the processed output, for generating a rate value, the rate value indicating a rate of peaks of the envelope bounding outputted readings of the processed output.

19. The deflection sensor of claim 18 wherein the rate processor is a Fast Fourier Transformer (FFT), a Discrete Fourier Transformer (DFT), or a Wavelet Transformer.

20. The deflection sensor of claim 19 wherein the rate is a respiration rate of a person lying on the optical fiber, wherein the person's breathing creates deflections of the optical fiber, or wherein the rate is a heart rate of a person lying on the optical fiber, wherein the person's heart beat creates deflections of the optical fiber.

21. A fiber-optic sensor comprising:
image processor means, receiving a speckle pattern from an image sensor, the speckle pattern created by interference of light passing through an optical fiber, wherein deflection of the optical fiber changes the speckle pattern, for outputting a current frame of pixels for each sample period, wherein the current frame is in a sequence of frames;
a baseline frame representing an array of pixel values;
pixel compare means for comparing pixels from the current frame to corresponding pixels in the baseline frame to generate pixel differences;
absolute means for generating absolute pixel differences which are absolute values of the pixel differences from the pixel compare means;
sum means for summing the absolute pixel differences from the absolute means to generate a sum-of-the-absolute differences (SAD), the SAD being an over-baseline value that indicates a magnitude of differences between the speckle pattern of the current frame and a speckle pattern of the baseline frame; and
output means for outputting the over-baseline value for each current frame in the sequence of frames as a processed output.
